(19) United States
(12) Patent Application Publication
(10) Pub. No.: US 2026/0043399 A1
(43) Pub. Date: Feb. 12, 2026

(54) WIRELESS PERISTALTIC PUMP

(71) Applicant: VANDERBILT UNIVERSITY, Nashville, TN (US)

(72) Inventor: Xiaoguang Dong, Nashville, TN (US)

(21) Appl. No.: 19/286,487

(22) Filed: Jul. 31, 2025

Related U.S. Application Data
(60) Provisional application No. 63/681,900, filed on Aug. 12, 2024.

(51) Int. Cl. F04B 43/12; A61F 5/00

(52) U.S. Cl. CPC F04B 43/12; A61F 5/0059; A61F 5/0066

(57) ABSTRACT

The present disclosure provides for wirelessly actuated soft robotic undulating pumps designed to efficiently transport both viscous fluids and solid cargos in human patients, methods of manufacturing a peristalsis-producing stent, and systems including a pair of soft magnetic sheets and a magnetic actuator configured to produce a magnetic field which generates complementary undulation of the pair of soft magnetic sheets. In an aspect, the pair of soft magnetic sheets can have at least a first soft magnetic sheet and a second soft magnetic sheet, and wherein a magnetization profile of the first soft magnetic sheet is coordinated with a magnetization profile of the second soft magnetic sheet.

### CROSS-REFERENCE TO RELATED APPLICATIONS

This application claims the benefit of U.S. Provisional application entitled “WIRELESS PERISTALTIC PUMP” and having Ser. No. 63/681,900, filed Aug. 12, 2024, which is herein incorporated by reference in its entirety.

### BACKGROUND

The transport of fluids and solids is a vital process inside the human body, facilitated by the wave-like motion in the lumen called peristalsis. However, peristalsis may be compromised due to tumor growth, resulting in difficulties in lumen motility. The dysmotility of the human lumen can result in blockages and pose numerous challenges, including aspiration in the lungs and reproductive issues in the female oviduct. Restoring peristalsis in medical devices, such as medical stents, can prevent device blockage and promote effective transport.

### SUMMARY

In accordance with the purpose(s) of this disclosure, as embodied and broadly described herein, the disclosure, in various aspects, relates to wireless peristaltic pumps for transporting viscous fluids and solid cargos in confined spaces and methods of use thereof.

Aspects of the present disclosure provide for a wirelessly actuated soft robotic undulating pump designed to efficiently transport both viscous fluids and solid cargos in human patients. Embodiments of the present disclosure include: an apparatus with a tubular member having a radially external side and a radially internal side, at least a first magnetic soft sheet disposed on the radially internal side of the tubular member, and at least a second magnetic soft sheet disposed on the radially internal side of the tubular member, wherein a magnetization profile of the first magnetic soft sheet is coordinated with a magnetization profile of the second magnetic soft sheet. The apparatus can have a phase difference between a magnetization phase of the first magnetic soft sheet and a magnetization phase of the second magnetic soft sheet, where the phase difference is approximately (¾)π. The apparatus can further include a magnetic actuator which, when actuated, causes the first magnetic soft sheet and the second magnetic soft sheet to produce coordinated out of phase undulation. The tubular member can be a silicone stent for placement in peristalsis-controlled passageways. In some examples, the magnetization profile of the first magnetic soft sheet and the magnetization profile of the second magnetic soft sheet are configured such that a magnetic actuator can cause both the first magnetic soft sheet and the second magnetic soft sheet to produce a net peristaltic flow from an entry end to an exit end of the tubular member. In some examples, the first magnetic soft sheet comprises a first plurality of magnetic modules joined together in a first flexible sheet, and the second magnetic soft sheet comprises a second plurality of magnetic modules joined together in a second flexible sheet. Each of the first flexible sheet and the second flexible sheet can comprise an elastic membrane. Each of the first plurality of magnetic modules and each of the second plurality of magnetic modules can be formed from a polymer matrix including ferromagnetic particles.

Next, a method of manufacturing a peristalsis-producing stent is provided, the method comprising the steps of embedding a first plurality of magnetic modules in a first flexible sheet and a second plurality of magnetic modules in a second flexible sheet. The method further includes bonding at least the first flexible sheet and at least the second flexible sheet to a radially internal side of a stent and actuating at least the first flexible sheet and at least the second flexible sheet with a magnetic actuator to produce peristaltic waves.

The method can further include magnetizing at least a first plurality of magnetic modules and at least a second plurality of magnetic modules such that the first plurality of magnetic modules has a first magnetic phase, and the second plurality of magnetic modules has a second magnetic phase, wherein the first magnetic phase has a phase difference from the second magnetic phase. The phase difference between the first magnetic phase and the second magnetic phase can be in the range of approximately 0 to approximately (¾)π. In some examples, magnetizing at least the first plurality of magnetic modules and at least the second plurality of magnetic modules further comprises wrapping the first plurality of magnetic modules and the second plurality of magnetic modules around a cylindrical fixture of an impulse magnetizer, the impulse magnetizer having a magnetic field of approximately 2.3 T. The method can further include laser-cutting a magnetic composite sheet to form at least the first plurality of magnetic modules and at least the second plurality of magnetic modules.

In some examples, the method can include arranging the first plurality of magnetic modules in a first line for magnetization and arranging the second plurality of magnetic modules in a second line for magnetization. Embedding the first plurality of magnetic modules in the first flexible sheet and the second plurality of magnetic modules in the second flexible sheet can further comprise arranging the first plurality of magnetic modules in side-by-side order on the first flexible sheet and the second plurality of magnetic modules in side-by-side order on the second flexible sheet, bonding the first plurality of magnetic modules in the first flexible sheet and the second plurality of magnetic modules in the second flexible sheet, and applying a hydrogel coating to each of the first flexible sheet and the second flexible sheet.

Additionally, a system is provided, the system comprising a pair of soft magnetic sheets, where the pair of soft magnetic sheets having at least a first soft magnetic sheet and a second soft magnetic sheet, and wherein a magnetization profile of the first soft magnetic sheet is coordinated with a magnetization profile of the second soft magnetic sheet. The system further includes a magnetic actuator configured to produce a magnetic field which generates complementary undulation of the pair of soft magnetic sheets. In some examples, a phase difference between a magnetization phase of the first soft magnetic sheet and a magnetization phase of the second soft magnetic sheet is approximately (¾)π. The system can further include a tubular member having a radially internal side and a radially external side, wherein the pair of soft magnetic sheets is bonded to the radially internal side of the tubular member. In some examples, the first soft magnetic sheet comprises a first plurality of magnetic modules joined together in a first flexible sheet, and the second soft magnetic sheet comprises a second plurality of magnetic modules joined together in a second flexible sheet. The first magnetization profile and the second magnetization profile can be configured such that the magnetic actuator can cause both the first soft magnetic sheet and the second soft magnetic sheet to produce a net flow from an entry end to an exit end of the tubular member. In some examples, the magnetic actuator comprises a rotating Halbach array of a plurality of magnets.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1D show an illustration of the concept and design of a wirelessly actuated undulating pump and its integration into an esophageal stent according to various embodiments of the present disclosure. FIG. 1A shows a concept of an esophageal stent with integrated magnetic soft sheets in the upper gastrointestinal tract for restoring peristalsis. A rotating external magnetic field is applied to actuate the magnetic soft sheets. FIG. 1B shows an illustration of the undulating motion of a magnetic sheet within a period driven by a rotating external magnetic field. FIG. 1C shows sequential optical images of a pair of magnetic soft sheets transporting solid spheres (scale bar, 5 mm). FIG. 1D shows an illustration of an esophageal stent integrated with the magnetic soft sheets transporting both liquids and solids (scale bars, 5 mm).

FIGS. 2A-2G show an illustration of a fabrication process and characterization of the magnetic soft sheet according to various embodiments of the present disclosure. FIG. 2A shows an illustration of fabricating the magnetic modules by cutting a thin sheet of magnetic composite using a UV laser machine. FIG. 2B shows an illustration of magnetizing the magnetic modules inside an impulse magnetizer (IM-10-30, ASC Scientific). FIG. 2C shows an illustration of assembling the magnetic modules into a magnetic soft sheet with the assistance of a fixture made of a Polyimide tape. FIG. 2D is an illustration of bonding the magnetized modules to an elastic membrane made of Ecoflex 00-30 with a thickness of 200 μm. FIG. 2E is an illustration of coating a thin layer of Polyethylene Glycol Diacrylate (PEGDA) hydrogel on the magnetic sheet to reduce adhesion. FIG. 2F shows images of the uncoated and coated sheets with measured water contact angles (104 and 65 degrees) (scale bar, 500 μm). FIG. 2G shows an illustration of assembling multiple magnetic sheets into a cylindrical pump.

FIGS. 3A-3H show an illustration of a single magnetic soft sheet according to various embodiments of the present disclosure. FIG. 3A shows an illustration of the magnetization profile of a magnetic soft sheet in a circular array. FIG. 3B shows an illustration of the magnetization and dimension of a magnetic module. FIGS. 3C and 3D show the magnetization phase (FIG. 3C) and magnitude (FIG. 3D) profiles for a magnetic soft sheet. FIG. 3E shows an illustration of the magnetic actuation setup for the experiments (FIGS. 3F and 3G). FIG. 3F shows video frames of a magnetic soft sheet under a rotating magnetic field in the x-z plane. FIG. 3G shows video frames of a magnetic sheet under a rotating magnetic field in the x-y plane. FIG. 3H shows the extracted kinematics of the magnetic soft sheet shown in FIG. 3G.

FIGS. 4A-4H show a characterization of a pair of magnetic soft sheets for pumping according to various embodiments of the present disclosure. FIG. 4A shows an illustration of a pair of magnetic soft sheets. FIG. 4B shows extracted kinematics of a pair of magnetic soft sheets with a phase shift of Δϕ=3π/4. FIG. 4C shows video frames of transporting liquid (syrup) by a pair of magnetic soft sheets. FIG. 4D shows the average liquid transporting speed as a function of the phase shift Δϕ. FIG. 4E shows the liquid transporting speed as function of sheet spacing. FIG. 4F shows video frames of transporting a solid sphere (hydrogel bead) by a pair of magnetic soft sheets. FIG. 4G shows the average bead transporting speed as a function of the phase shift between two neighboring sheets. FIG. 4H shows the average bead transporting speed as a function of sheet spacing.

FIGS. 5A-5G show a characterization of the performance of transporting liquid and solid cargos by a cylindrical pump according to various embodiments of the present disclosure. FIG. 5A shows video frames of three magnetic sheets (phase shift: Δϕ=3π/4) pumping in glycerol visualized by green dye. FIG. 5B shows video frames of pumping liquid (syrup) inside a silicone tube. FIG. 5C shows the average transporting speed of mucus and pure syrup using the magnetic soft sheets inside a tube. FIG. 5D shows the average transporting speed of syrup when the cylindrical pump is placed at different angles. FIG. 5E shows video frames of pumping hydrogel spheres by the cylindrical pump. FIG. 5F shows the average transporting speed of hydrogel spheres in different diameters by the cylindrical pump. FIG. 5G shows the average transporting speed of hydrogel spheres (diameter: 4.5 mm) when the cylindrical pump is placed at different angles.

FIGS. 6A-6H show a demonstration of integrating magnetic soft sheets inside a metal stent for pumping liquids and solids according to various embodiments of the present disclosure. FIG. 6A shows an optical image of the nine magnetic soft sheets. FIG. 6B shows an optical image of the assembled metal esophageal stent with integrated magnetic soft sheets. FIG. 6C shows an optical image of the assembled esophageal stent with marked dimensions. FIG. 6D shows experimental images of the metal esophageal stent with integrated magnetic soft sheets and the experimental setup used for testing. FIG. 6E shows video frames of the metal stent transporting liquid (syrup, dynamic viscosity: 5000 mPa s) and hydrogel spheres inside an esophagus phantom. FIG. 6F shows an X-ray image of a metal esophageal stent inside the phantom. FIG. 6G shows video frames of transporting multiple hydrogel pieces by the stent. FIG. 6H shows video frames of transporting a large hydrogel sphere by the stent.

FIGS. 7A and 7B show a characterization of the magnetic field waveform according to various embodiments of the present disclosure. FIG. 7A shows an illustration of the measurement of the magnetic field. FIG. 7B shows an example measurement of the magnetic field at a given location (d=3.7 cm).

FIGS. 8A-8D are an example experimental setup for magnetically actuating the stent according to various embodiments of the present disclosure.

FIG. 9 is a depiction of pumping speed difference for a pair of sheets with or without hydrogel coating according to various embodiments of the present disclosure.

FIG. 10 is an example flow wake for a silicone stent with magnetic sheets pumping glycerol while visualized by green dye according to various embodiments of the present disclosure.

FIG. 11 is an illustration of transportation of porcine mucus by a pair of magnetic soft sheets according to various embodiments of the present disclosure.

FIG. 12 is an example estimation of the magnetic field generated by neighboring modules and sheets according to various embodiments of the present disclosure.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, biomedical engineering and mechanical engineering techniques and the like, which are within the skill of the art. Such techniques are explained fully in the literature.

The following examples are put forth to provide those of ordinary skill in the art with a complete disclosure and description of how to perform the methods and use the compositions and compounds disclosed and claimed herein. Efforts have been made to ensure accuracy with respect to numbers (e.g., amounts, measurements, etc.), but some errors and deviations should be accounted for.

Before the embodiments of the present disclosure are described in detail, it is to be understood that, unless otherwise indicated, the present disclosure is not limited to particular materials, machines, computing processes, or the like, as such can vary. It is also to be understood that the terminology used herein is for purposes of describing particular embodiments only and is not intended to be limiting. It is also possible in the present disclosure that steps can be executed in different sequence where this is logically possible.

It should be noted that ratios, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

### Discussion

Disclosed are various approaches for a wirelessly actuated soft robotic undulating pump designed to efficiently transport both viscous fluids and solid cargos in human patients. The transport of fluids and solids is a vital process inside the human body, facilitated by the wave-like motion in the lumen called peristalsis. However, peristalsis may be compromised due to tumor growth, resulting in difficulties in lumen motility. The dysmotility of the human lumen can result in blockages and pose numerous challenges, including aspiration in the lungs and reproductive issues in the female oviduct. While medical devices, such as medical stents, can serve to open these biological passageways, these devices can also become blocked. Restoring peristalsis in medical devices, such as medical stents, can prevent device blockage and promote effective transport.

Here, a wirelessly actuated soft robotic undulating pump designed to efficiently transport both viscous fluidic and solid cargos is proposed. The pump can have multiple undulating soft sheets which can be actuated by magnetic fields. The kinematics of a single sheet and the coordination between pairs of sheets are systematically designed to generate undulation and peristalsis, enabling the pumping of both liquids and solids. In some embodiments, the undulating pump can be integrated into medical devices (e.g., an esophageal stent). The same undulating motion-based pumping mechanism can be adapted for usage in other organs, such as the female oviduct, thereby offering potential applications for treating lumen dysmotility in various diseases. The proposed wirelessly actuated robotic pumping mechanism holds promise in facilitating diverse implantable medical devices aimed at treating diseases characterized by impaired peristalsis and dysmotility.

Accordingly, various embodiments of the present disclosure are directed to systems and methods for using a wirelessly actuated soft robotic undulating pump designed to efficiently transport both viscous fluids and solid cargos in human patients. The proposed wireless undulating motion-based pump mechanism is generic for various medical stents to restore the muscular transport inside the lumens of the human body. For example, the fallopian tubes, also known as the oviduct, are part of the female reproductive system and play a crucial role in transporting eggs from the ovaries to the uterus. The peristalsis motion of the fallopian tube muscle may also be impaired in pelvic inflammatory disease due to genetic issues which also cause issues of transporting eggs. The same magnetic soft sheets could be integrated into medical stents inside the fallopian tube for transporting eggs from the ovaries to the uterus. In addition, it could also be integrated with airway stents for transporting excessive mucus. Therefore, by emulating and restoring the natural peristalsis motion, the proposed mechanism and device could address the current challenge of lumen dysmotility.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

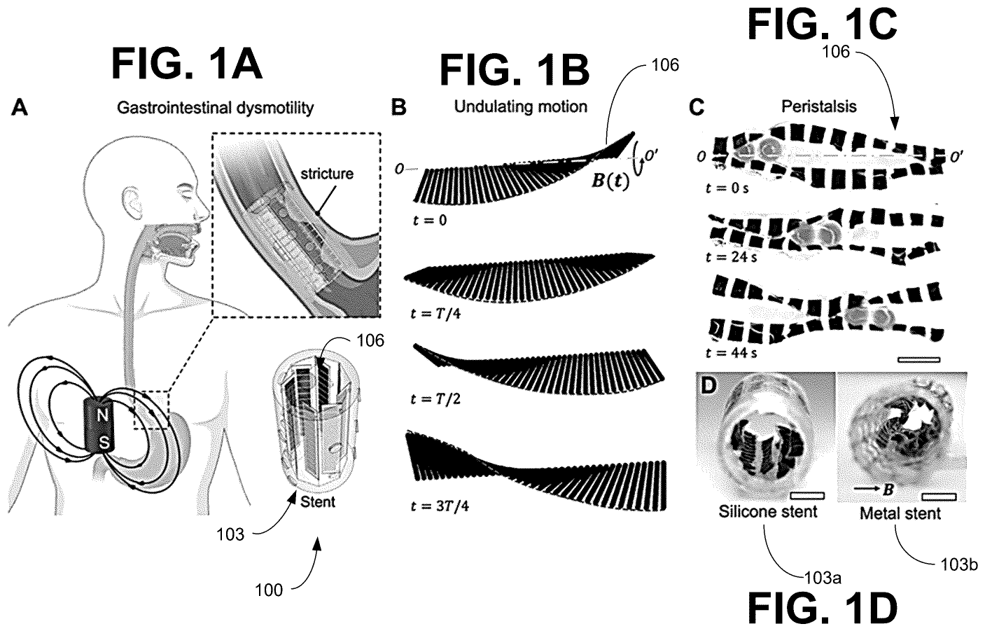

With reference to FIGS. 1A-1D, shown is an example concept of the wirelessly actuated undulating pump 100 and its integration into an esophageal stent 103. FIG. 1A shows a concept of an esophageal stent with integrated magnetic soft sheets 106 in the upper gastrointestinal tract for restoring peristalsis. A rotating external magnetic field is applied to actuate the magnetic soft sheets 106. FIG. 1B shows an illustration of the undulating motion of a magnetic sheet 106 within a period driven by a rotating external magnetic field. The magnetic field is rotating about the fixed long edge of the magnetic soft sheet 106. One long edge of the magnetic sheet 106 is fixed while the other edge is free. FIG. 1C shows sequential optical images of a pair of magnetic soft sheets 106 transporting solid spheres (scale bar, 5 mm). FIG. 1D shows an illustration of an esophageal stent 103 integrated with the magnetic soft sheets 106 transporting both liquids and solids (scale bars, 5 mm).

As shown in FIG. 1A, when integrated into a stent 103, the wirelessly actuated undulating pump 100 can include a tubular member (e.g., the stent 103) having a radially external side and a radially internal side, representing the inside surface and the outside surface of the tubular member. The tubular member can be a silicone stent 103a or a metal mesh stent 103b (FIG. 1D) for placement in peristalsis-controlled passageways. In some examples, the tubular member can be another form of medical device for placement in peristalsis-controlled passageways. The wirelessly actuated undulating pump 100 can include at least a first magnetic soft sheet 106 disposed on the radially internal side of the tubular member. In some examples, the wirelessly actuated undulating pump 100 includes at least a second magnetic soft sheet 106 disposed on the radially internal side of the tubular member. As shown in FIGS. 1A-1D, some examples include a plurality of magnetic soft sheets 106 disposed on the internal side of the tubular member. The plurality of magnetic soft sheets 106 can include an even number of magnetic soft sheets 106 to provide the pair-wise coordinated undulation shown in FIG. 1C.

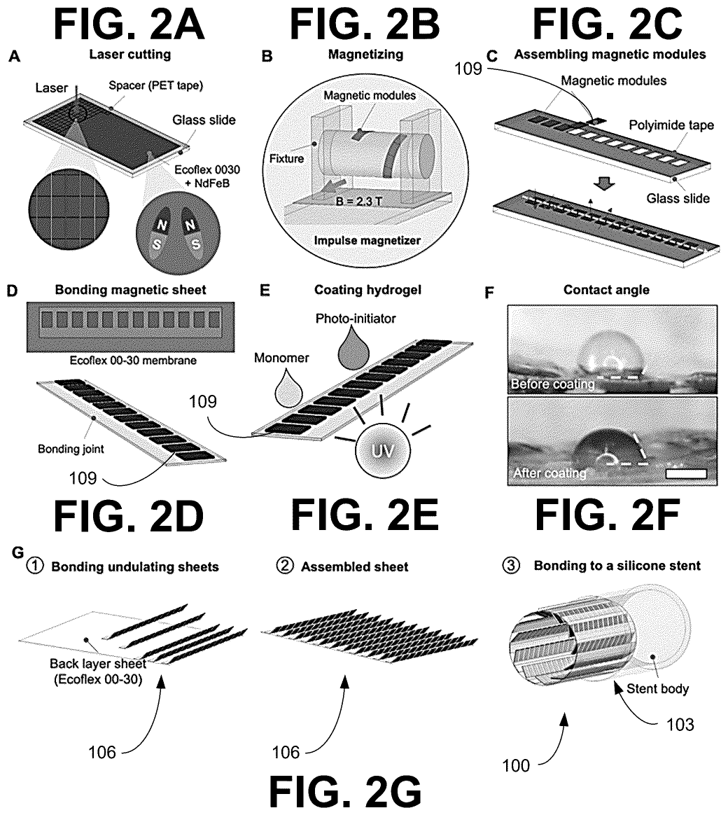

Moving on to FIGS. 2A-2G, shown is an illustration of an example fabrication and characterization of the magnetic soft sheet 106. FIG. 2A shows an illustration of fabricating the magnetic modules 109 by cutting a thin sheet of magnetic composite using a UV laser machine. In some examples, the thin sheet is made of NdFeB particles and Ecoflex 00-30 (weight ratio: 2 to 1) with thickness controlled by a Polyester tape as a spacer. FIG. 2B shows an illustration of magnetizing the magnetic modules 109 inside an impulse magnetizer (IM-10-30, ASC Scientific). The magnetic modules 109 are wrapped around a cylinder to encode the desired magnetization profiles. FIG. 2C shows an illustration of assembling the magnetic modules 109 into a magnetic soft sheet 106 with the assistance of a fixture made of a Polyimide tape. Each magnetic module 109 can be approximately 3 mm by 1.5 mm by 0.2 mm. However, other sizes are also possible. FIG. 2D is an illustration of bonding the magnetized modules 109 to an elastic membrane made of Ecoflex 00-30 with a thickness of 200 μm. FIG. 2E is an illustration of coating a thin layer of Polyethylene Glycol Diacrylate (PEGDA) hydrogel on the magnetic sheet 106 to reduce adhesion. FIG. 2F shows images of the uncoated sheets and coated sheets 106 with measured water contact angles (104 and 65 degrees) (scale bar, 500 μm). FIG. 2G shows an illustration of assembling multiple magnetic sheets 106 into a cylindrical pump (silicone stent 103a).

As shown in FIGS. 2A-2G, the magnetic soft sheets 106 can comprise a plurality of magnetic modules 109 joined together in a flexible sheet. The flexible sheet can comprise an elastic membrane, a polymer sheet, a silicone sheet, or some other form of biologically-compatible and highly-flexible material. The magnetic modules 109 can be formed from a polymer matrix including ferromagnetic particles. In some examples, the plurality of magnetic modules 109 can be arranged in a substantially uniform distribution in the flexible sheet to form the magnetic soft sheet 106. Each magnetic module 109 in the plurality of magnetic modules 109 that forms a magnetic soft sheet 106 can have a progressively different magnetization profile. By having different magnetization profiles for each magnetic module 109, when a magnetic field is applied to the magnetic soft sheet 106, each magnetic module 109 will react to the magnetic field in a different manner. Thus, by magnetizing the plurality of magnetic modules 109 as described above, the application of a magnetic field can produce the wave-like undulating motion in a magnetic soft sheet 106.

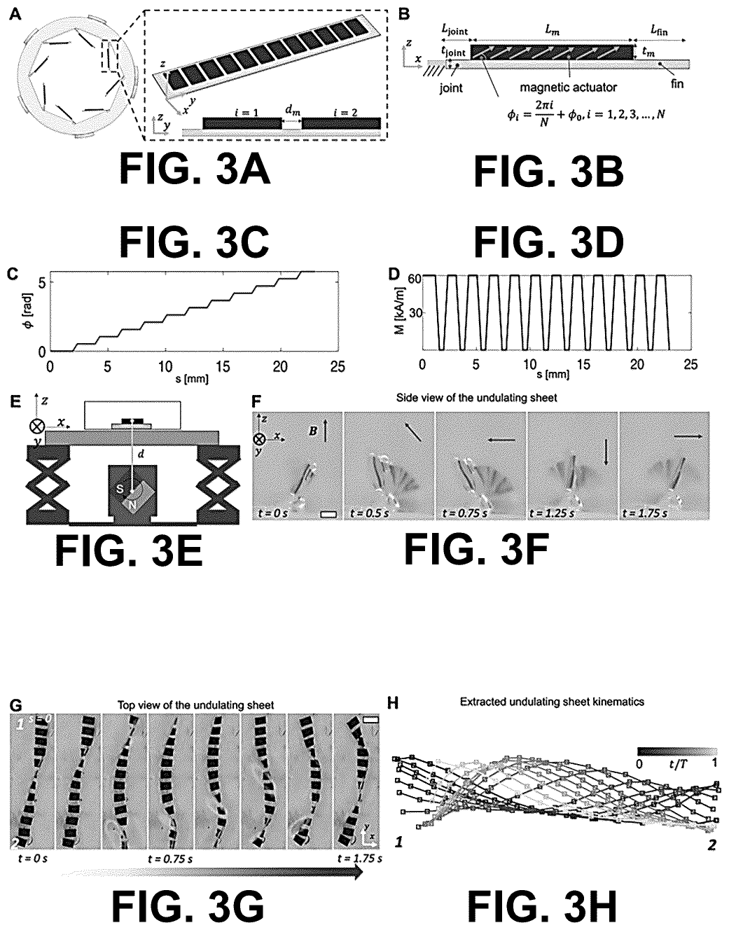

Turning now to FIGS. 3A-3H, shown is an example design and characterization of a single magnetic soft sheet 106. FIG. 3A shows an illustration of the magnetization profile of a magnetic soft sheet 106 in a circular array. For each sheet, the magnetization phase profile is defined as ϕi(s), s∈(0, L]. FIG. 3B shows an illustration of the magnetization and dimension of a magnetic module 109. FIGS. 3C and 3D show the magnetization phase (FIG. 3C) and magnitude (FIG. 3D) profiles for a magnetic soft sheet 106. FIG. 3E shows an illustration of the magnetic actuation setup for the experiments (FIGS. 3F and 3G). FIG. 3F shows video frames of a magnetic soft sheet 106 under a rotating magnetic field in the x-z plane. For the external magnetic field: average magnitude B=55 mT, frequency f=0.5 Hz. FIG. 3G shows video frames of a magnetic sheet 106 under a rotating magnetic field in the x-y plane. For the external magnetic field: average magnitude B=55 mT, f=0.5 Hz. FIG. 3H shows the extracted kinematics of the magnetic soft sheet 106 shown in FIG. 3G. In both FIGS. 3F and 3G, the liquid is glycerol (dynamic viscosity: 983 mPa s).

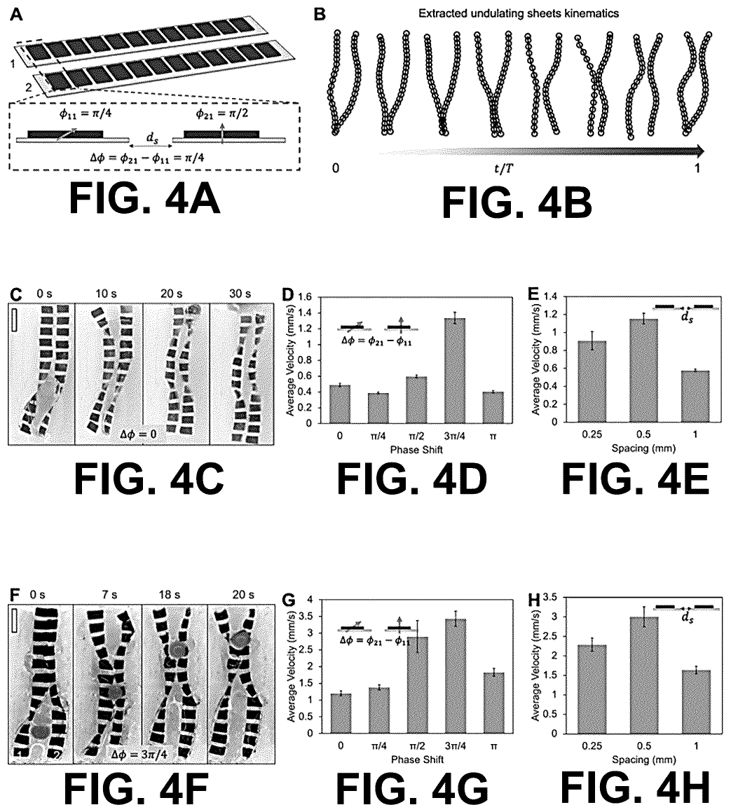

Moving on to FIGS. 4A-4H, shown is an example characterization of a pair of magnetic soft sheets 106 for pumping. FIG. 4A shows an illustration of a pair of magnetic soft sheets 106. The phase shift between two magnetic soft sheets is defined as the angle difference of the magnetic moments of the front magnetic modules Δϕ=ϕ21−ϕ11. The sheet spacing is denoted as ds. FIG. 4B shows extracted kinematics of a pair of magnetic soft sheets with a phase shift of Δϕ=3π/4. FIG. 4C shows video frames of transporting liquid (syrup) by a pair of magnetic soft sheets. Phase shift Δϕ=0. ds=0.5 mm. Average B=55 mT, f=2 Hz. FIG. 4D shows the average liquid transporting speed as a function of the phase shift Δϕ. Magnetic field: B=55 mT, f=2 Hz. FIG. 4E shows the liquid transporting speed as function of sheet spacing. In FIGS. 4C-E, the liquid is syrup with a dynamic viscosity of 5000 mPa s. FIG. 4F shows video frames of transporting a solid sphere (hydrogel bead) by a pair of magnetic soft sheets. Phase shift Δϕ=3π/4, ds=0.5 mm. Magnetic field: B=55 mT, f=2 Hz. FIG. 4G shows the average bead transporting speed as a function of the phase shift between two neighboring sheets. FIG. 4H shows the average bead transporting speed as a function of sheet spacing. In FIGS. 4F-H, the diameter of the hydrogel bead is 3.5 mm. In FIGS. 4F-H, the liquid for lubrication is glycerol. Error bar indicates standard deviation for n=5 trials. In all figures, scale bars, 5 mm.

In some examples, a magnetization profile of the first magnetic soft sheet 106 in a pair of magnetic soft sheets 106 is coordinated with a magnetization profile of the second magnetic soft sheet 106. In some examples, the phase difference between a magnetization phase of the first magnetic soft sheet 106 and a magnetization phase of the second magnetic soft sheet 106 is approximately (¾)π. However, the phase difference can be in the range of approximately 0 to approximately π. In some examples, the magnetization profile of the first magnetic soft sheet 106 and the magnetization profile of the second magnetic soft sheet 106 are configured such that a magnetic actuator can cause both the first magnetic soft sheet 106 and the second magnetic soft sheet 106 to produce a net peristaltic flow from an entry end to an exit end of the tubular member (e.g., stent 103).

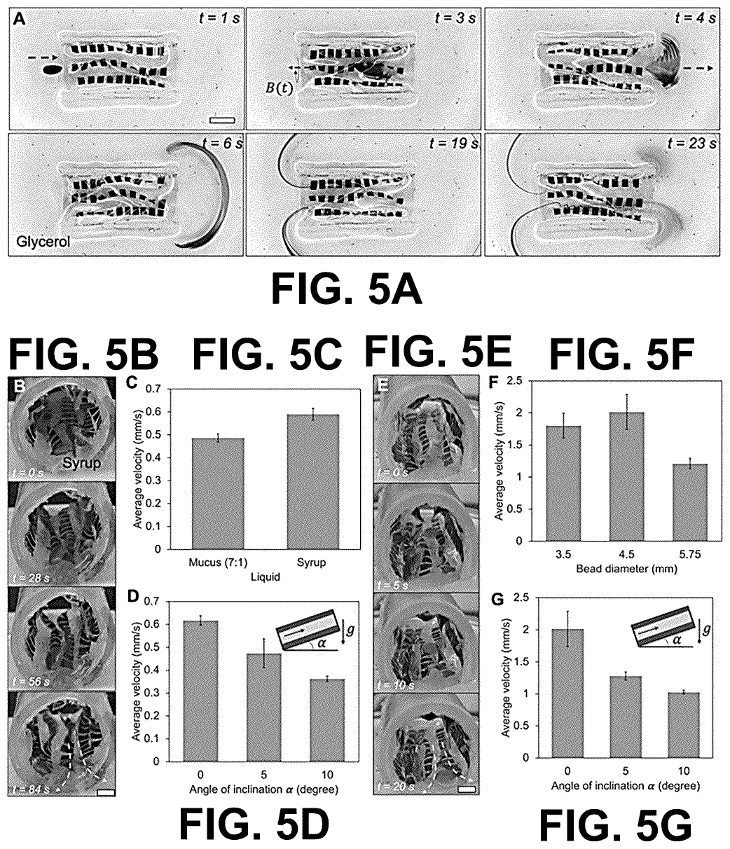

Next, at FIGS. 5A-5G, shown is an example characterization of the performance of transporting liquid and solid cargos by a cylindrical pump 100. FIG. 5A shows video frames of three magnetic sheets 106 (phase shift: Δϕ=3π/4) pumping in glycerol visualized by green dye. FIG. 5B shows video frames of pumping liquid (syrup) inside a silicone tube 103. FIG. 5C shows the average transporting speed of mucus and pure syrup using the magnetic soft sheets 106 inside a tube 103. FIG. 5D shows the average transporting speed of syrup when the cylindrical pump 100 is placed at different angles. FIG. 5E shows video frames of pumping hydrogel spheres by the cylindrical pump 100. FIG. 5F shows the average transporting speed of hydrogel spheres in different diameters by the cylindrical pump 100. Glycerol is used as a lubricant liquid. FIG. 5G shows the average transporting speed of hydrogel spheres (diameter: 4.5 mm) when the cylindrical pump 100 is placed at different angles. In all experiments, the magnetic field is B=55 mT, f=2 Hz. In all experiments, the dynamic viscosities of syrup and mucus are 5000 mPas and 11 307 mPa s, respectively. Error bars indicate standard deviation for n=5 trials. Scale bars, 5 mm.

A magnetic actuator can be used to cause the first magnetic soft sheet 106 and the second magnetic soft sheet 106 to produce coordinated out of phase undulation. In some examples, the magnetic actuator comprises a rotating Halbach array of a plurality of magnets.

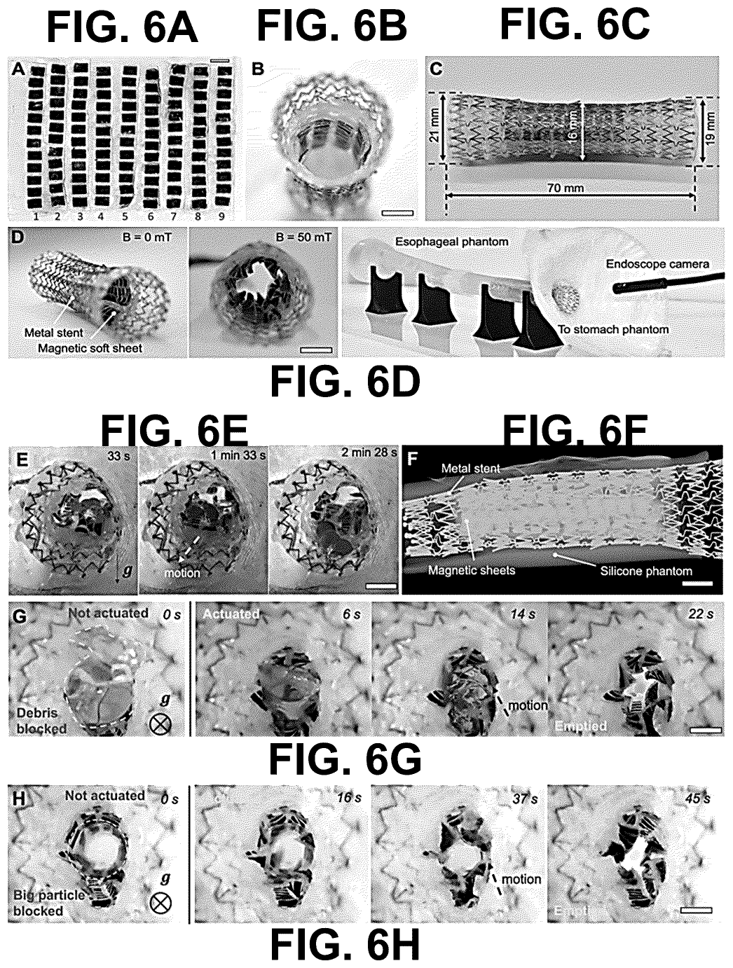

In FIGS. 6A-6H, shown is an example demonstration of integrating magnetic soft sheets 106 inside a metal stent 103 for pumping liquids and solids. FIG. 6A shows an optical image of the nine magnetic soft sheets 106. Each sheet can have twelve magnetic modules 109. However, in some examples, each sheet can have any number of magnetic modules 109, limited only by the size of the magnetic module 109 and the size of the stent 103. FIG. 6B shows an optical image of the assembled metal esophageal stent 103 with integrated magnetic soft sheets 106. A stent 103 can include as few as two magnetic soft sheets 106, but preferably contain enough magnetic soft sheets 106 to provide peristaltic motion over the internal surface area of the stent 103. FIG. 6C shows an optical image of the assembled esophageal stent 103 with marked dimensions. As depicted in FIG. 6C, the dimensions of the example stent 103 are 21 mm in diameter at a first end, 16 mm in diameter near the middle of the stent 103, 19 mm in diameter at a second end of the stent 103, and a length of 70 mm. However, other sizes can be arranged depending on the desired end location of the stent 103. For example, a fallopian stent would have different dimensions than an esophageal stent 103.

FIG. 6D shows experimental images of the metal esophageal stent 103 with integrated magnetic soft sheets 106 and the experimental setup used for testing. An external magnetic field of an average magnitude of ≈55 mT is applied. The system includes a magnetic actuation system, the esophagus phantom, the metal stent 103 with integrated magnetic soft sheets 106, and an endoscope camera. FIG. 6E shows video frames of the metal stent 103 transporting liquid (syrup, dynamic viscosity: 5000 mPa s) and hydrogel spheres inside an esophagus phantom. FIG. 6F shows an X-ray image of a metal esophageal stent inside the phantom. FIG. 6G shows video frames of transporting multiple hydrogel pieces by the stent. FIG. 6H shows video frames of transporting a large hydrogel sphere by the stent. Scale bars, 5 mm.

A peristalsis-producing stent can be manufactured by embedding a plurality of magnetic modules 109 in one or more flexible sheets 106 as described above. The plurality of magnetic modules 109 can first be formed from laser-cutting a magnetic composite sheet to form the magnetic modules 109. After the magnetic modules 109 are cut, they can be embedded in the flexible sheets. In some examples, the magnetic modules 109 can be arranged into lines for magnetization and embedding into the flexible sheets 106. In some examples, the magnetic modules 109 can be arranged side-by-side on a flexible sheet 106, bonded to the flexible sheet 106, and the coated in a hydrogel coating.

The magnetic modules 109 of the flexible sheets 106 can be magnetized by wrapping the sheet 106 having the magnetic modules 109 around a cylindrical fixture of an impulse magnetizer. In some examples, the impulse magnetizer has a magnetic field of approximately 2.3 T. The magnetic modules 109 of each sheet 106 can be magnetized such that the magnetic modules 109 of one sheet 106 have a different magnetic phase than the magnetic modules 109 of the next sheet 106. The difference between the magnetic phases can be referred to as the phase difference. In some examples, the phase difference between the magnetic phase of one sheet 106 and the magnetic phase of another sheet 106 is in the range of approximately 0 to approximately (¾)π. Once the magnetic modules 109 have been magnetized, each of the flexible sheets 106 can then be bonded to a radially internal side of the stent 103 or other hollow tubular member.

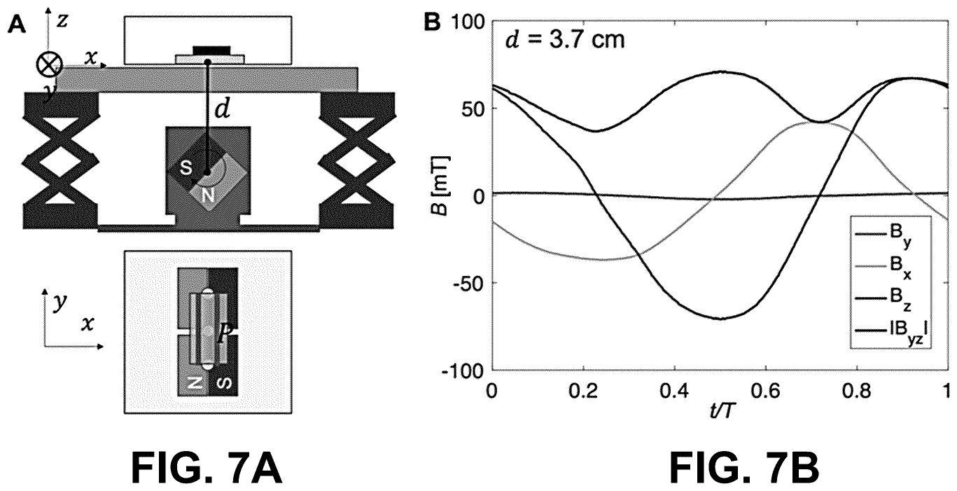

In FIGS. 7A and 7B, shown is an example characterization of the magnetic field waveform. FIG. 7A shows an illustration of the measurement of the magnetic field. FIG. 7B shows an example measurement of the magnetic field at a given location (d=3.7 cm).

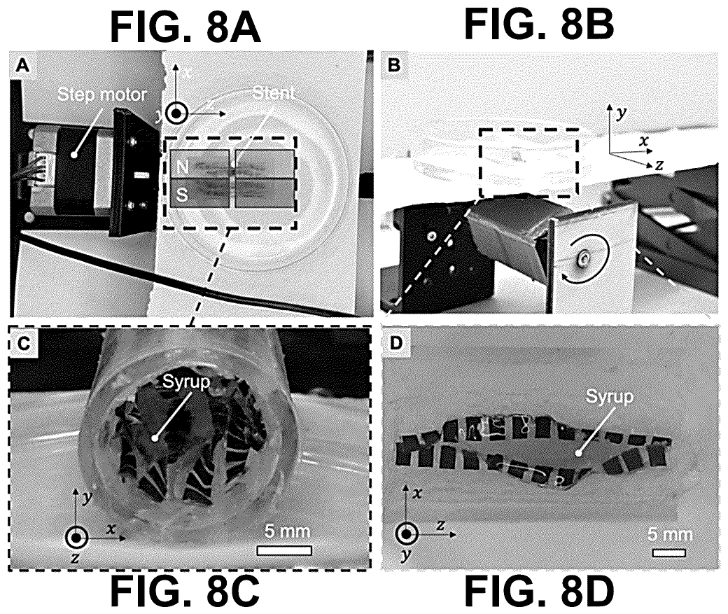

FIGS. 8A-8D show an experimental setup for magnetically actuating the stent. FIG. 8A shows an optical image of the experimental setup in a top view. FIG. 8B shows an optical image of the experimental setup in a perspective view when testing the pair-wise coordination of two magnetic sheets. FIG. 8C shows a zoomed-in optical image of a silicone stent with magnetic soft sheets integrated for pumping syrup. FIG. 8D shows an optical image of a pair of magnetic soft sheets pumping syrup.

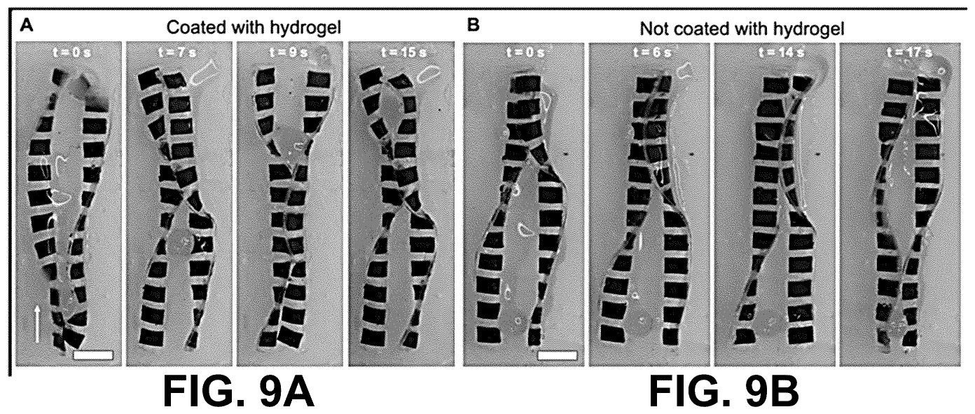

Moving to FIGS. 9A and 9B, shown is an example of pumping speed difference for a pair of sheets with or without hydrogel coating. FIG. 9A shows pair-wise transportation of hydrogel sphere with coated magnetic sheets. FIG. 9B shows pair-wise transportation of hydrogel sphere with non-coated magnetic sheets. Scale bars, 5 mm.

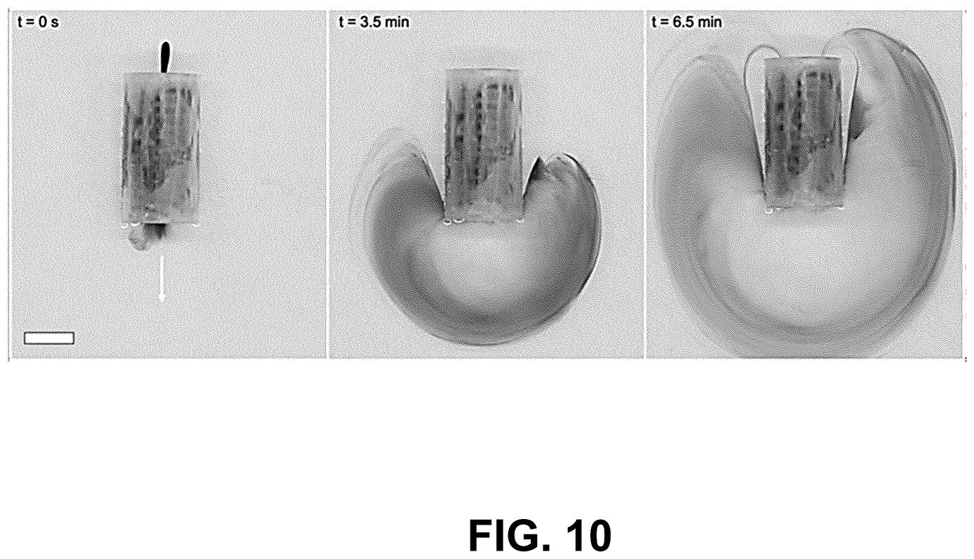

Next, at FIG. 10, shown is an example flow wake for a silicone stent with magnetic sheets pumping glycerol while visualized by green dye. The stent is placed in a horizontal plane inside a container filled with viscous fluid (pure glycerol, dynamic viscosity: 983 mPa·s). White arrow indicates the pumping direction. Scale bar, 10 mm.

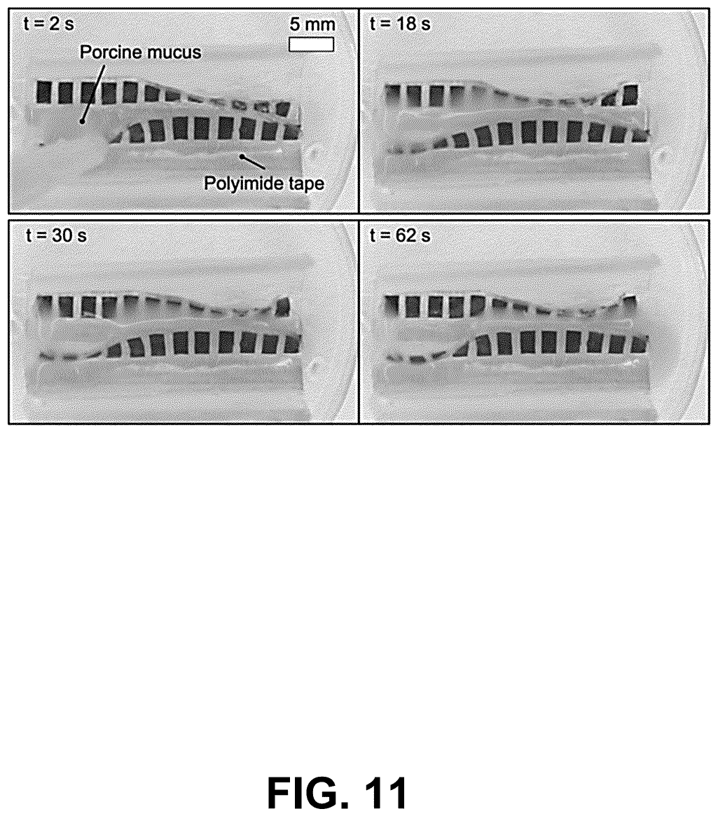

FIG. 11 shows Transportation of porcine mucus by a pair of magnetic soft sheets. Phase shift Δϕ=−3π/4.

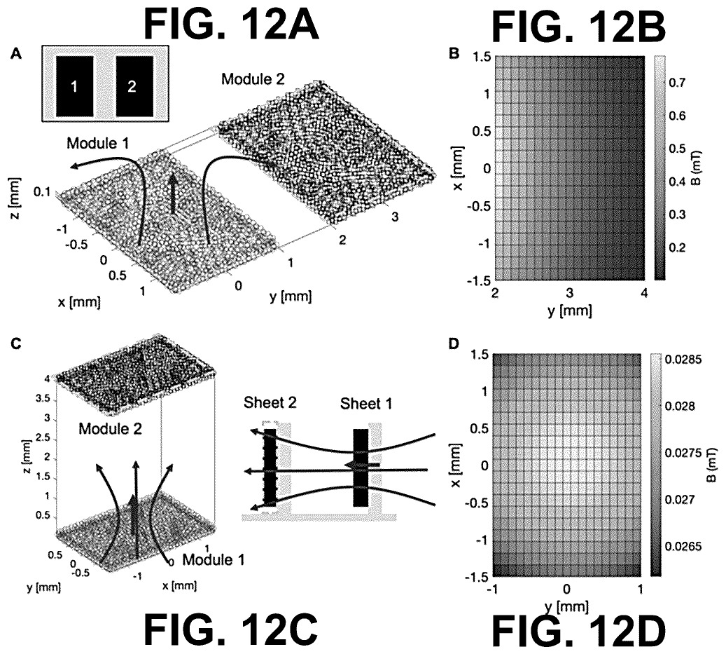

Finally, in FIG. 12A-12D, shown is an example the estimation of the magnetic field generated by neighboring modules and sheets. FIG. 12A shows an illustration of the magnetic field generated at the location of a magnetic module by a neighboring magnetic module. FIG. 12B shows a magnetic field distribution using a distributed magnetic dipole model for the configuration in FIG. 12A. FIG. 12C shows an illustration of the magnetic field generated at the location of a magnetic module by the closest magnetic module in a neighboring sheet. FIG. 12D shows a magnetic field distribution using a distributed magnetic dipole model for the configuration in FIG. 12C.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

### EXAMPLE A

### 1. Introduction

Within the human body, the transport of viscous fluids and solids plays a pivotal role in various biological processes facilitated by peristalsis within luminal structures like the esophagus, small intestine, and female oviduct. Peristalsis is a wave-like movement of the muscles in the human lumen to efficiently move viscous fluids and solid cargos. The impairment of peristalsis in various diseases can result in luminal occlusion. Particularly, tumors in the esophagus may grow and obstruct the lumen, hindering the passage of food and liquids. Particularly, esophageal adenocarcinoma is rising in prevalence and primarily affects the aging population, with the median age of patients diagnosed between 65 and 74 years old, and over 30% of cases occurring in individuals over the age of 75 in the United States. In other diseases, such as achalasia, the dysmotility of the esophagus leads to food blockage when the esophageal muscles fail to contract properly, preventing the downward movement of food toward the stomach.

Treatment often involves the usage of a silicone or metal tube known as an esophageal stent to maintain an open passage in the narrowed portion of the esophagus, to allow the swallowing of solids and liquids. However, challenges arise when food particles or other materials accumulate around the stent, leading to blockages, and in some cases aspiration when the food enters the airway. This obstruction may cause the ingested material to divert into the airways instead of progressing into the stomach, which poses a significant concern, particularly for individuals with compromised respiratory function. It can result in respiratory complications such as pneumonia. Therefore, restoring peristalsis is crucial for the effective transport of liquids and solids to address these challenging medical conditions.

The development of a peristalsis-based soft robotic pump holds promise for mimicking and restoring peristalsis. Compact and wirelessly actuated soft robotic pumps designed for transporting cargos inside medical stents could potentially overcome these challenges. Soft robotic pumps, powered by various soft actuators such as fluidic and pneumatic actuators, electropneumatic, dielectric elastomer actuator, and electrodynamic effect been shown before but have not been demonstrated to integrate into medical stents due to several fundamental challenges. First, soft robotic pumps, propelled by fluidic or pneumatic means, face the hurdle of requiring bulky and tethered external compressors, posing integration challenges for medical stents. Second, although electrically driven pumps show promise with their compact size, the batteries required for onboard power and actuation currently have severely limited lifetimes. Additionally, the incorporation of flexible electronics to regulate actuator motion while maintaining resilience and conformity to curved surfaces presents significant challenges in circuit design and fabrication.

In contrast, external actuation methods such as acoustic, magnetic actuation, and chemical actuation, offer a noninvasive method to actuate soft structures to pump fluids by penetrating biological tissues deep inside the body. Among them, magnetic actuation could penetrate deep inside the organs and provides larger mechanical forces and torques when combined with ferromagnetic or ferrimagnetic materials. Existing works have shown arrays of millimeter- or micrometer-scale magnetic soft actuators for transporting microparticles in liquids. However, the current pumping mechanism is limited to lab-on-a-chip fluid transport and have not been shown to be integrated into medical stents. In addition, the design and control of magnetic structures that allow efficient pumping mechanism for viscous fluids and solids remains unclear.

To tackle this challenge, a magnetically actuated soft robotic pump is proposed that can restore peristalsis through the incorporation of magnetically actuated soft sheets within the stent structure. Wireless actuation using external magnetic fields eliminates the need for bulky on-board components, ensuring the safe interaction of the soft robotic structure with esophageal tissue. This example systematically investigates both single sheet pumping and pair-wise coordination of the magnetic soft sheets to optimize the pumping efficiency. The soft robotic pump could transport viscous fluids, particle-liquid suspensions, biofluids such as mucus, and solid spheres of different sizes. Finally, this example demonstrates integrating the undulating pump into a silicone and metal stent for the esophagus to generate artificial peristalsis to aid in propelling liquids and solids inside an esophageal phantom. The active pumping function is shown to reduce the risks of the blockage of the lumen in the control experiments. The same pumping mechanism can be potentially used for patients with diseases of dysmotility in various lumens inside the body. The proposed mechanism of restoring the natural peristalsis motion thus paves the way for the next-generation robotic medical devices to improve the quality of life.

### 2. Results

### 2.1. Concept, Design, and Fabrication of the Undulating Motion-Based Pump

The undulating motion of a magnetic soft sheet plays a fundamental role in pumping liquid and solid cargos in the proposed wireless soft robotic pump. As shown in FIG. 1A, the objective is to produce peristalsis through programming an array of magnetic soft sheets and controlling their coordinated movements using external magnetic fields. As illustrated in FIG. 1B, the application of a rotating magnetic field B (t) induces the undulating motion in each sheet. A magnetic soft sheet consists of magnetic modules created by blending ferromagnet particles (NdFeB, average diameter: 5 μm) in a polymer matrix, and magnetized in a specific magnetization profile (see FIGS. 2A-2G and “Experimental Section” for the fabrication process). The distributed magnetic torque induced along the sheet long axis OO′ creates the undulating motion by bending the magnetic modules along the axis OO′ of the sheet. When subjected to an external magnetic field, a pair of magnetic sheets will have coordinated motion like peristalsis for transporting particles.

The pairs of magnetic soft sheets are further integrated into a medical stent to allow transporting liquid and solid cargos as illustrated in FIG. 1C. By integrating the magnetic soft sheets in a circular array within a stent, e.g., a hollow tube, it serves a dual purpose: providing radial support to the lumen in cases of stricture while simultaneously restoring the peristalsis motion for pumping. Particularly, an esophageal stent is designed to assist in the digestion of large food pieces, thereby preventing blockages and aspirations. The coordination between neighboring sheets and the surface of the hollow tube creates a peristalsis motion, showcased for transporting liquids and solids. The wireless actuation capability of the magnetic soft sheets allows for minimally invasive cargo transport. Notably, magnetically actuated devices have been reported before with wireless actuation and small sizes in lab-on-a-chip applications and miniature soft robots that can swim or crawl for minimally invasive medical operations. However, magnetically actuated soft robotic pumps that can restore peristalsis and seamlessly integrated with medical stents have not been reported before.

In FIGS. 2A-2G, the method of fabricating the magnetic soft sheets is further introduced. The fabrication process includes preparing the magnetic modules, programming their magnetization, and assembling them into magnetic soft sheets, and integrating into a medical stent (see Materials and Methods for the details). Briefly, first, the magnetic modules (dimensions: 3 mmby 2 mm by 0.2 mm) are prepared by cutting a rectangular magnetic sheet made of NdFeB micromagnets and Ecoflex 00-30 by a laser machine as shown in FIG. 2A. Second, the magnetic modules in a magnetic soft sheet are assembled into a strip in a head-to-tail manner and magnetized by wrapping the strip around a cylindrical fixture in an impulse magnetizer with a magnetic field of 2.3 T as illustrated in FIG. 2B. Third, the magnetized magnetic modules are assembled in a side-by-side manner with the assistance of an assembly jig made from a polyimide tape (FIG. 2C). Fourth, the magnetic modules are further bonded to a thin layer of Ecoflex 00-30 and released from the assembly jig (FIG. 2D). Lastly, the assembled structure could be further coated with other materials such as PDMS and hydrogel for biocompatibility and hydrophilic surface property to facilitate liquid wetting. FIG. 2E demonstrates one example of coating Polyethylene GlycolDiacrylate (PEGDA) hydrogel on the surface of the device. The water contact angle is significantly reduced from 104 degrees to 65 degrees after the hydrogel coating that allows better wetting of water-based liquid as a lubrication layer as shown in FIG. 2F. Multiple magnetic sheets that are obtained with the same method are further bonded to a thin back layer made of Ecoflex 00-30 and then wrapped into a tube to be further bonded (FIG. 2G) to the inside surface of an elastic cylindrical tube, a silicone stent, and other meshed metal stents.

### 2.2. Single Magnetic Sheet Undulating Motion

The undulating motion of a single magnetic sheet is achieved by patterning its magnetization profile. As shown in FIG. 3A, the magnetic soft sheet is designed to have multiple magnetic modules with a rotational angular offset encoded between neighboring magnetic moments in the x-z plane. FIG. 3B shows that the angle of the magnetic moment Mi for the i-th (i=1, . . . , N) magnetic module is given by ϕi=2πi N+ϕ0, where ϕ0 is the initial phase of the magnetic module. Mi is then given by Mix=Mcosϕi, Miz=Msinϕi (magnetization magnitude: M=61.9 kA m−1). The magnetization phase and magnitude profiles given in FIGS. 3C and 3D are essential to produce the undulating motion. When a rotating magnetic field B(t) is applied in the x-z plane, each magnetic module experiences a magnetic torque that bends the module at an angle. The magnetic torque applied on each magnetic module is given by τi=VmMi×B(t) where Vm is the volume of the magnetic module. There is also fluid drag applied on the sheet that is depending on the rotating frequency of the magnetic field f and the dynamic viscosity of the liquid μ. The bending motions of the neighboring magnetic modules are coupled due to the connection with non-magnetic materials. The bending angle is shifted across different magnetic modules along the sheet's long axis. Jointly they create an undulating motion that travels along the sheet as the magnetic field rotates in clockwise or counterclockwise in the x-z plane. Compared with other types of undulating pumps, the reported magnetic sheets are fully soft, wirelessly actuated, and have a relatively good programmability in terms of the pair-wise coordination by jointly designing neighboring sheets.

To further demonstrate the undulating motion, FIGS. 3E-H showcase a magnetic sheet submerged inside glycerol when being actuated by a rotating magnetic field with an average magnitude of B=55 mT, and frequency of f=2 Hz (FIGS. 7A and 7B). A rotating permanent magnet (50 mm by 25 mm by 25 mm, NdFeB, N45) is used to actuate the magnetic sheet as shown in FIG. 3E (see FIGS. 8A-8D for the experimental setup). Subsequently, FIGS. 3F and 3G show the optical images of the magnetic soft sheet in a side view and top view, respectively. Traveling wave motions are clearly shown in both the top view and the side view. Further extraction of the curve of the sheet edge shows the quantitative characterization of the traveling wave like motion as shown in FIG. 3H, when the peak value of the curve is shifting along the y-axis in a period. The extracted curves show that a peak shift from one end “1” to the other end “2”, indicating a traveling wave propagating as the magnetic module bends sequentially with a phase lag. The phase lag is fully programmable by designing the magnetization of each magnetic module. Lastly, with the flexible connection between neighboring magnetic modules, the traveling wave is induced that is essential for the efficient propulsion of liquids and solids.

### 2.3. Pair-Wise Sheet Coordination for Pumping Liquids and Solids

This example further shows the kinematics of a pair of magnetic sheets by varying their phase difference in FIGS. 4A-4H. Two magnetic sheets could be designed differently to induce coordinated motion for generating peristalsis. The coordinated motion is due to the time lag between the two neighboring sheets with different magnetization profiles undergoing undulating motion. Although the two magnetic sheets experience similar temporal magnetic torques and undulating motion, the phase in their motion depends on both the planted orientation and their magnetization profile when actuated by the same magnetic field. The two magnetic sheets have different magnetization phase ϕ11 and ϕ21 as shown in FIG. 4A. With a constant phase difference Δϕ=ϕ21−ϕ11 (e.g., Δϕ=3π/4) for all magnetic modules, pair-wise coordination is induced as shown in FIG. 4B. The extracted kinematics in FIG. 4B shows that a narrow part is traveling when rotating the external magnetic field, which creates peristalsis.

To further investigate the pumping performance of a pair of magnetic sheets of different designs, the liquid and solid pumping speeds were studied when varying the phase difference and spacing of a pair of magnetic sheets. FIG. 4C shows that a liquid droplet (syrup, dynamic viscosity: 5000 mPa s) with a volume of ≈1 mL is effectively transported by a pair of magnetic sheets from one side to the other within 30 s. In FIG. 4D, shown is a further comparison of the liquid droplet pumping speed by several different pairs of magnetic sheets which have a phase difference Δϕ from 0 to 3π/4. The pair of sheets with Δσ=3π/4 allow the maximum average transporting speed of ≈1.3 mm s−1. The pair-wise motion in this case resembles the peristalsis when a narrow part between the two sheets is traveling along the long axis of the two sheets. Moreover, FIG. 4E shows the average transporting speeds by magnetic soft sheet pairs with different spacings ds, which is defined as shown in FIG. 4A. For the sheets with a width of ≈3 mm, the maximum transporting speed is achieved when they have ds=0.5 mm and Δσ=3π/4. The magnetic interaction becomes negligible at a separation distance of ds=0.5 mm. This is because the magnetic field generated by a neighboring magnetic module typically registers <1 mT, notably lower than the magnetic field produced by external magnets, which typically measures ≈55 mT. When further decreasing the spacing to 0.25 mm, the transporting speed is deteriorated by the magnetic interaction between the two magnetic sheets. In contrast, when further increasing the spacing to 1 mm, the transporting speed is also reduced when the coordinated peristalsis motion between the two sheets becomes less efficient.

Meanwhile, to investigate the performance of pumping solids using a pair of magnetic soft sheets, magnetic sheets of different phase shifts and spacings are prepared to perform solid pumping experiments. FIG. 4F shows that a hydrogel solid sphere of a diameter of 3.5 mmis being transported from one end to the other when slightly lubricated by glycerol. The pair of sheets have Δϕ=3π/4 and a spacing of ds=1 mm. In addition, FIG. 4G shows that a pair of sheets with Δσ=3π/4 gives the maximum transporting speed consistent with that in the liquid droplet transporting experiments. Moreover, FIG. 4H shows that when the spacing is ds=0.5 mm, the pair of magnetic soft sheets give the maximum transporting speed as the two sheets could exert a relatively large pressure on the solid sphere to propel it forward. Lastly, the hydrogel layer coated on the magnetic sheets provides a lubrication layer that greatly enhances the pumping of solid cargos as shown in FIGS. 9A and 9B.

### 2.4. Demonstration of Integrating Magnetic Soft Sheets Inside a Silicone Tube for Pumping

The magnetic soft sheets with optimized phase difference and spacing are further integrated into a silicone tube as demonstrated in FIG. 5. With the optimized magnetic sheets in terms of both phase difference and spacing, ten magnetic sheets are integrated inside a tubular structure made of silicone to create a cylindrical pump for transporting liquids and solids. First, the fluid flow pattern is shown in FIG. 5A which demonstrates the efficient pumping of viscous liquid (glycerol, dynamic viscosity: 890 mPa s) visualized by green dyes. The wake pattern when the stent is fully submerged in glycerol is shown in FIG. 10. Second, FIG. 5B shows an investigation of the pumping speed for different types of liquids. Next, the pumping speed for syrup (dynamic viscosity: 5000 mPa s) and porcine mucus was tested by mixing porcine mucin with water according to a mixing ratio of 7:1 by weight. The porcine mucus has a viscosity of ≈11 300 mPa s at room temperature. In both cases, the cylindrical pump shows a relatively fast pumping speed with ≈0.5 and 0.6 mm s−1 for the syrup and mucus, respectively, as shown in FIG. 5C. The presence of biofluids such as mucus may allow better transportation as mucus is slippery as shown in FIG. 11. Lastly, the cylindrical pump was tilted to see if it can overcome gravity to pump the liquids upward effectively. FIG. 5D shows a reduced transporting speed for syrup when increasing the tilt angle from 0 to 10 degrees. The angle may be further increased when producing smaller and denser magnetic sheets inside the pump. Nonetheless, the demonstrated cylindrical pump inside a silicone stent shows promising applications of transporting viscous liquids.

Similarly, in FIGS. 5E-G, this example investigates the ability to pump solids of different sizes and at different tilting angles using the cylindrical pump. FIG. 5E shows that multiple hydrogel beads of different sizes are transported simultaneously. Moreover, FIG. 5F shows that the beads with a diameter of ≈4.5 mm are transported at the fastest speed due to the relatively large pressure applied on the beads by the magnetic sheets, while the peristalsis motion is not deteriorated due to the sheet-solid interaction. Lastly, FIG. 5G shows that the cylindrical pump can also pump solid spheres up to 10 degrees upward indicating its ability to overcome gravity for pumping solids.

### 2.5. Demonstration of Integrating Magnetic Soft Sheets Inside a Medical Stent for Pumping

To further show the potential for medical applications, the undulating pump is demonstrated to integrate into a commercial metal stent (16×70 ALIMAXX-ES Fully Covered Esophageal Stent, Merit Medical) as shown in FIGS. 6A-6H. Nine magnetic sheets with the optimized phase difference and spacing are integrated inside an esophageal stent as shown in FIGS. 6A-C. The stent is already covered with a polymer mesh as shown in FIG. 6B, which is for preventing stent tissue ingrowth. Built on that, the magnetic sheets are first wrapped into a cylindrical pump and then the cylindrical pump is further bonded to a metal stent (see Materials and Methods for details). The assembled meshed metal stent is deployed by squeezing it to fit inside a 3D-printed esophagus phantom as shown in FIG. 6D. The device is then demonstrated to restore the motility of the esophagus to effectively transport liquid and solid cargos, potentially useful for patients with esophageal cancer or stricture. FIG. 6E shows that the liquid and solid are transported inside an esophagus phantom after being deployed inside a phantom made of silicone (Elastic 50A, Formlabs). The process is visualized with an endoscope when a magnetic field of ≈55 mT and 2 Hz is applied. To deploy the metal stent with integrated magnetic sheets inside a human esophagus, endoscope procedures will be followed when the stent is delivered using the delivery tool used for delivering a conventional metal esophageal stent. The resilience of the magnetic soft sheets allows them to withstand the mechanical stress and still function during the deployment. Lastly, the stent could also be easily monitored by medical imaging such as X-ray imaging in addition to an endoscope. For example, X-ray imaging is used to monitor the magnetic soft sheets which are clearly visible as shown in FIG. 6F.

Finally, the stent is demonstrated to allow the transport of large solids to avoid blockage and therein aspiration. In contrast, a traditional esophageal stent has a high risk of food blockage due to the lack of peristalsis. FIGS. 6G and 6H prove the effectiveness with control experiments. This example shows the effectiveness of reducing blockage using the stent integrated with magnetic soft sheets. As shown in FIG. 6G, the hydrogel debris in irregular shapes initially block the cross-section of the stent when no magnetic field is applied on the magnetic sheets. The cluster of hydrogel debris starts being squeezed and pumped downward into the stomach phantom when a rotating magnetic field of ≈55 mT at 2 Hz is applied. This example further demonstrates the transport of large and intact hydrogel beads (diameter: 12 mm) using the stent with integrated magnetic sheets. As shown in FIG. 6H, without the artificial peristalsis, the spheres block the esophagus. In contrast, with the restored peristalsis, magnetic soft sheets integrated into the stent enable the large particles to be pumped into the stomach phantom within 45 s when a magnetic field (55 mT, 2 Hz) is applied.

### 3. Conclusion

In summary, this example has reported a soft robotic pump 100 that is fully wireless and can transport liquids and solids efficiently. The fundamental undulating motion is realized by programming a magnetic sheet 106 with a specific magnetization profile. Peristalsis motion is further encoded by designing different magnetization profiles in neighboring sheets 106. Different designs have been systematically investigated to optimize the pair-wise coordination for both liquid and solid pumping. Finally, this example has demonstrated creating an esophageal stent 103 as an example to restore the peristalsis for pumping liquids and solids to prevent blockage and therein aspiration. The proposed wirelessly actuated robotic pumping mechanism is generic and holds promise in enabling diverse implantable medical devices designed to address the challenges of lumen dysmotility in various diseases such as esophageal cancer. Compared with existing soft robotic pumps, the device is fully wireless which can be seamlessly integrated with existing medical stents. This proposed wireless soft robotic pump 100 marks a significant step toward the development of innovative solutions in the field of soft robotics and point-of-care medical devices.

The proposed undulating motion-based wireless pump 100 may be further improved. First, in some examples, the magnetic soft sheets 106 can be scaled down using advanced manufacturing methods such as micro-molding to further adapt to narrower lumens. The surface of the magnetic soft sheet 106 can be patterned with microstructures and omni-phobic material coatings for improving the transport of various fluids and solids. In addition, the current prototype is actuated with a permanent magnet mounted on a motorized rotational stage. Typically, a person can tolerate magnetic fields up to 7 Tesla, whereas the magnetic field measures ≈55 mT which is considerably lower and thus deemed safe. To facilitate efficient pumping, it is generally required to have a magnetic field ranging from 40 to 60 mT. In some examples, the magnetic field can be further minimized by increasing the magnetic moment of the magnetic module and reducing material elastic modulus. For future applications at home, the esophageal stent 103 can be actuated by a wearable magnetic actuation system which is developed for long-term actuation of the robotic esophageal stent 103.

In addition, the proposed wireless undulating motion-based pump 100 is generic for various medical stents 103 to restore the muscular transport inside the lumens of the human body. For example, the fallopian tubes, also known as the oviduct, are part of the female reproductive system and play a crucial role in transporting eggs from the ovaries to the uterus. The peristalsis motion of the fallopian tube muscle may also be impaired in pelvic inflammatory disease due to genetic issues which also cause issues of transporting eggs. The same magnetic soft sheets 106 can be integrated into medical stents 103 inside the fallopian tube for transporting eggs from the ovaries to the uterus. In addition, it can also be integrated with airway stents 103 for transporting excessive mucus. Therefore, by emulating and restoring the natural peristalsis motion, the proposed mechanism and device can address the current challenge of lumen dysmotility.

### 4. Experimental Section

Fabrication of Magnetic Modules and Back Layer: To fabricate the magnetic modules, NdFeB micromagnets were first mixed thoroughly with Ecoflex 00-30 (Smooth-on Inc.) with a ratio of 1:2 by weight. Then, four layers of Polyester (PET) tapes with a thickness of 65 μm for each layer were used as spacers to construct a thin wall on the edges of an acrylic substrate. The liquid mixture of the Ecoflex 00-30 and NdFeB micromagnets were spread out slowly to reduce the formation of air bubbles. A sharp razor blade was used to scrape the composite materials to ensure the uniform distribution of the mixture on the substrate. Subsequently, the substrate together with the mixture composite was cured at a temperature of 70° C. on a hot plate for 1 h. Lastly, a UV laser cutter (LPKF U4, LPKF AG) was used to cut the cured sheet into a 12 by 6 matrix of magnetic modules with the dimensions of 3 mm by 2 mm by 0.2 mm. Each matrix was cut using a “hinge” method with longitudinal deeper cuts and latitudinal shallower cuts to allow easy magnetization and assembly of the magnetic modules. Additionally, to fabricate the back layer, a separate acrylic glass with four layers of PET tape as boundaries on the edges was used as a substrate to spread out pure Ecoflex 00-30 uniformly and cured at a temperature of 70° C. on a hot plate for 1 h.

Magnetizing of the Magnetic Modules: To magnetize the modules, a strip of magnetic modules (12 modules) cut by the laser machine were first wrapped around a magnetizing fixture consisting of a cylinder fixed between two side walls bonded to a glass slide. The fixture was a simple cylinder marked with a π/4 angular increment and a double-sided adhesive tape layered on the outside of the cylinder to allow the strip to adhere onto it. The fixture was bonded on a glass slide using double-sided adhesive tape to secure the orientation of the fixture. When wrapping the strip around the cylinder, the magnetic modules were placed to not overlap with each other, ensuring proper magnetization of the array. Subsequently, this fixture was placed inside an impulse magnetizer (IM-10-30, ASC Scientific) and a magnetic field impulse of 2.3 T was then applied at a maximum discharging voltage of 300 V. Each subsequent strip was placed on the cylinder with an angular increment on the cylinder to induce a desired incremental phase shift.

Assembly of the Magnetic Soft Sheets: The magnetized modules with the desired orientation were bonded to the back layer made of cured Ecoflex 00-30. A 1-mm distance was maintained between neighboring modules while the modules were aligned with the longer edge parallel to each other. Uncured Ecoflex 00-30 was used as an adhesive to bond the magnetic modules to the Ecoflex 00-30 back layer. Once all the magnetic modules were bonded to the Ecoflex 00-30 back layer, the sheet 106 was cut out according to a desired dimension of the joint and the fin. For example, a width of 0.5 mm was selected for both the joint and the fin. In addition, a 4-mm wide extra back layer was kept, extending the joint to further allow secured bonding of the sheet 106 onto the base layer.

Integration of Magnetic Soft Sheets on a Silicone Stent: Multiple magnetic sheets 106 were first bonded onto a base layer to form a magnetic sheet 106 blanket by bonding the sheets 106 using uncured Ecoflex 00-30. The blanket was then rolled into a magnetic sheet cylinder with the two edges bonded using uncured Ecoflex 00-30. After curing, the cylindrical structure was tested by being compressed into a flat structure to check for durability. Upon ensuring the bonding strength of all the magnetic sheets 106 on the cylindrical base layer, freshly prepared Ecoflex 00-30 was applied onto the inner surface of the prepared silicone stent. The prepared magnetic sheet cylinder was subsequentially compacted and inserted into the stent. The compression on the magnetic sheet cylinder was removed once the cylinder was inside the stent 103. All the gaps between the magnetic sheet cylinder and the stent inner surface were ensured to have uncured Ecoflex 00-30 by compression. Lastly, the assembled stent 103 was heated to complete the bonding.

Integration of Magnetic Sheets on a Meshed Metal Stent: Similar to “Integration of magnetic soft sheets on a silicone stent,” magnetic soft sheets 106 were bonded to a meshed metal stent 103 (16×70 ALIMAXX-ES Fully Covered Esophageal Stent, Merit Medical) while ensuring the back layer integrity. As the metal stent 103 had a varying diameter that was thinner in the middle and wider at the two ends, glycerol was applied for lubrication when placing the magnetic sheet cylinder inside. In the process, 0.1-0.2 mL of glycerol helped lubricate the inner surface and allow for an easier decompression and installation of the magnetic sheet cylinder. Subsequently, a spatula was used to apply sufficient Ecoflex 00-30 first starting from the middle and then working out toward the edges to further seal the edges. After curing the Ecoflex 00-30 at a temperature of 70° C. on a hot plate for 30 min, the durability of the assembled stent 103 was checked by performing the stent compression test.

Preparation of an Esophagus Phantom: The esophagus phantom was prepared by 3D-printing the segmented parts of a human upper gastrointestinal model. The phantom model was imported and segmented in Autodesk MeshMixer (Autodesk Research) and Fusion360 (Autodesk Research). The segmented parts were printed using a 3D printer (Form 3+, Formlabs Inc.) with UV curable resin Elastic 50A. The 3D printed parts were assembled and bonded by applying UV curable resin Elastic 50A under UV exposure.

Magnetic Actuation Setup: The magnetic actuation setup was composed of a step motor (NEMA 17) controlled by a step motor driver (L298N, HiLetgo) using an embedded controller (Arduino Uno). Two neodymium-iron-boron magnets (1 inch by 1 inch by 1 inch, N52) were fixed in a 3D printed fixture using Polylactic acid (PLA) and rotated about the metal shaft connected to the 3D-printed fixture for generating a rotating magnetic field with an average amplitude B ≈55 mT and frequency f from 0.1 to 2 Hz.

Preparation of Viscous Liquids and Solid Cargos: The viscous liquids used included syrup, glycerol, and porcine mucus. To make the porcine mucus, mucin (Chem-impex International Inc.) was mixed with water according to a weight ratio of 7 to 1. The viscosities of the liquids were measured by a rheometer (Bonvoisin Digital Rotary Viscometer). The solid cargos used were hydrogel spheres with different diameters (3-5 mm).

Therefore, the following is claimed:

1. An apparatus, comprising:
a tubular member having a radially external side and a radially internal side; and
at least a first magnetic soft sheet disposed on the radially internal side of the tubular member; and
at least a second magnetic soft sheet disposed on the radially internal side of the tubular member, wherein a magnetization profile of the first magnetic soft sheet is coordinated with a magnetization profile of the second magnetic soft sheet.

2. The apparatus of claim 1, wherein a phase difference between a magnetization phase of the first magnetic soft sheet and a magnetization phase of the second magnetic soft sheet is approximately (¾)π.

3. The apparatus of claim 1, further comprising a magnetic actuator which, when actuated, causes the first magnetic soft sheet and the second magnetic soft sheet to produce coordinated out of phase undulation.

4. The apparatus of claim 1, wherein the tubular member is a silicone stent for placement in peristalsis-controlled passageways.

5. The apparatus of claim 1, wherein the magnetization profile of the first magnetic soft sheet and the magnetization profile of the second magnetic soft sheet are configured such that a magnetic actuator can cause both the first magnetic soft sheet and the second magnetic soft sheet to produce a net peristaltic flow from an entry end to an exit end of the tubular member.

6. The apparatus of claim 1, wherein:
the first magnetic soft sheet comprises a first plurality of magnetic modules joined together in a first flexible sheet; and
the second magnetic soft sheet comprises a second plurality of magnetic modules joined together in a second flexible sheet.

7. The apparatus of claim 6, wherein each of the first flexible sheet and the second flexible sheet comprises an elastic membrane.

8. The apparatus of claim 6, wherein each of the first plurality of magnetic modules and each of the second plurality of magnetic modules is formed from a polymer matrix including ferromagnetic particles.

9. A method of manufacturing a peristalsis-producing stent, comprising:
embedding the first plurality of magnetic modules in a first flexible sheet and the second plurality of magnetic modules in a second flexible sheet;
bonding at least the first flexible sheet and at least the second flexible sheet to a radially internal side of a stent; and
actuating at least the first flexible sheet and at least the second flexible sheet with a magnetic actuator to produce peristaltic waves.

10. The method of claim 9, further comprising magnetizing at least a first plurality of magnetic modules and at least a second plurality of magnetic modules such that the first plurality of magnetic modules has a first magnetic phase and the second plurality of magnetic modules has a second magnetic phase, wherein the first magnetic phase has a phase difference from the second magnetic phase.

11. The method of claim 10, wherein the phase difference between the first magnetic phase and the second magnetic phase is in the range of approximately 0 to approximately (¾)π.

12. The method of claim 10, wherein magnetizing at least the first plurality of magnetic modules and at least the second plurality of magnetic modules further comprises wrapping the first plurality of magnetic modules and the second plurality of magnetic modules around a cylindrical fixture of an impulse magnetizer, the impulse magnetizer having a magnetic field of approximately 2.3 T.

13. The method of claim 9, further comprising laser-cutting a magnetic composite sheet to form at least the first plurality of magnetic modules and at least the second plurality of magnetic modules.

14. The method of claim 13, further comprising:
arranging the first plurality of magnetic modules in a first line for magnetization; and
arranging the second plurality of magnetic modules in a second line for magnetization.

15. The method of claim 9, wherein embedding the first plurality of magnetic modules in the first flexible sheet and the second plurality of magnetic modules in the second flexible sheet, further comprises:
arranging the first plurality of magnetic modules in side-by-side order on the first flexible sheet and the second plurality of magnetic modules in side-by-side order on the second flexible sheet;
bonding the first plurality of magnetic modules in the first flexible sheet and the second plurality of magnetic modules in the second flexible sheet; and
applying a hydrogel coating to each of the first flexible sheet and the second flexible sheet.

16. A system, comprising:
a pair of soft magnetic sheets, the pair of soft magnetic sheets having at least a first soft magnetic sheet and a second soft magnetic sheet, wherein a magnetization profile of the first soft magnetic sheet is coordinated with a magnetization profile of the second soft magnetic sheet; and
a magnetic actuator configured to produce a magnetic field which generates complementary undulation of the pair of soft magnetic sheets.

17. The system of claim 16, wherein a phase difference between a magnetization phase of the first soft magnetic sheet and a magnetization phase of the second soft magnetic sheet is approximately (¾)π.

18. The system of claim 16, further comprising a tubular member having a radially internal side and a radially external side, wherein the pair of soft magnetic sheets is bonded to the radially internal side of the tubular member.

19. The system of claim 16, wherein:
the first soft magnetic sheet comprises a first plurality of magnetic modules joined together in a first flexible sheet; and
the second soft magnetic sheet comprises a second plurality of magnetic modules joined together in a second flexible sheet.

20. The system of claim 18, wherein the first magnetization profile and the second magnetization profile are configured such that the magnetic actuator can cause both the first soft magnetic sheet and the second soft magnetic sheet to produce a net flow from an entry end to an exit end of the tubular member.
